(19) United States
(12) Patent Application Publication
(10) Pub. No.: US 2026/0047855 A1
(43) Pub. Date: Feb. 19, 2026

(54) LESION CROSSING CATHETER WITH OSCILLATING TIP

(71) Applicant: Shockwave Medical, Inc., Santa Clara, CA (US)

(72) Inventor: Hoa D. NGUYEN, San Jose, CA (US)

(73) Assignee: Shockwave Medical, Inc., Santa Clara, CA (US)

(21) Appl. No.: 18/808,874

(22) Filed: Aug. 19, 2024

(51) Int. Cl. A61B 17/22

(52) U.S. Cl. CPC A61B 17/22004

(57) ABSTRACT

A catheter includes an elongate body, a rotatable shaft disposed within the elongate body, an impactor forming a distal end of the catheter and configured to translate relative to the elongate body for impacting an occlusion in the body lumen, and an actuator configured to translate the impactor. The actuator includes a cam and cam follower operatively coupled to the rotatable shaft such that rotation of the rotatable shaft causes relative rotation and translation between the cam and cam follower. The impactor is coupled to the cam or the cam follower such that the impactor translates in conjunction with the cam or the cam follower to impact the occlusion in the body lumen.

### FIELD OF THE DISCLOSURE

The present disclosure relates generally to the field of medical devices and methods, and more specifically to catheter devices for treating lesions in body lumens, such as calcified lesions and occlusions in vasculature and kidney stones in the urinary system.

### BACKGROUND

A wide variety of catheters have been developed for treating calcified lesions, such as calcified lesions in vasculature associated with arterial disease. For example, treatment systems for percutaneous coronary angioplasty or peripheral angioplasty use angioplasty balloons to dilate a calcified lesion and restore normal blood flow in a vessel. In these types of procedures, a catheter carrying a balloon is advanced into the vasculature along a guide wire until the balloon is aligned with calcified plaques. The balloon is then pressurized (normally to greater than 10 atm), causing the balloon to expand in a vessel to push calcified plaques back into the vessel wall and dilate occluded regions of vasculature.

More recently, the technique and treatment of intravascular lithotripsy (IVL) has been developed, which is an interventional procedure to modify calcified plaque in diseased arteries. The mechanism of plaque modification is through use of a catheter having one or more acoustic shock wave generating sources located within a liquid that can generate acoustic shock waves that modify the calcified plaque. IVL devices vary in design with respect to the energy source used to generate the acoustic shock waves, with two exemplary energy sources being electrohydraulic generation and laser generation.

When treating an occlusion, a physician often crosses the occlusion (e.g., passes through the occluded area) and then feeds an angioplasty balloon and/or other tools down the artery to the blockage to perform the desired procedure. In some instances, however, such as the case of a chronic total occlusion (“CTO”) or a resistant fibrotic lesion, the occlusion may be so tight and solid that it is difficult to pass the treatment device into the true lumen of the distal vessel. Further, conventional guide wires may have difficulty penetrating thick, fibrous lesions, and may risk trauma to blood vessels when navigating narrow and tortuous regions of vasculature. Some physicians may implement atherectomy procedures (e.g., laser-based, mechanically cutting or shaving, mechanically rotating devices, etc.) to form a channel in a lesion in combination with an angioplasty balloon treatment, but many atherectomy devices and systems carry a higher risk of vessel perforation or vessel dissection as compared with a basic angioplasty balloon catheter. Even if the initial puncture of a lesion is successful, placement of dilation devices, like angioplasty balloons, can be very difficult in chronically occluded vessels. This makes the treatment of resistant lesions a technically challenging procedure that requires a long learning curve for interventional cardiologists. Accordingly, there is a need for a catheter device that can penetrate and treat resistant fibrotic and calcified lesions, such as CTOs, while minimizing the risk of trauma to blood vessels. Similar devices are needed for treating occlusions formed in other parts of the body, for example, kidney stones in the urinary system.

### SUMMARY

A lesion crossing catheter having an oscillating impactor at its distal end for delivering mechanical forces directly to an occlusion in a body lumen and methods of using a catheter to apply impacting forces to an occlusion are described herein. The impactor may translate forward and backward (i.e., advance in a distal direction and then return in a proximal direction) in an oscillatory manner to deliver repeated mechanical impact forces to an occlusion. The impactor may be oscillated by a cam assembly that is driven by a motor positioned in a handle of a catheter.

According to an aspect, a catheter for treating an occlusion in a body lumen includes an elongate body, a rotatable shaft disposed within the elongate body, an impactor forming a distal end of the catheter and configured to translate relative to the elongate body for impacting an occlusion in the body lumen, and an actuator configured to translate the impactor, the actuator including a cam and cam follower operatively coupled to the rotatable shaft such that rotation of the rotatable shaft causes relative rotation and translation between the cam and cam follower, wherein the impactor is coupled to the cam or the cam follower such that the impactor translates in conjunction with the cam or the cam follower to impact the occlusion in the body lumen.

Optionally, the catheter includes an enclosure surrounding at least a portion of the elongate body. Optionally, the impactor is coupled to the cam and translates in conjunction with the cam. Optionally, the cam is coupled to the impactor via a coupler. Optionally, the catheter includes a motor operable to rotate the shaft. Optionally, the shaft is coupled to the motor via a gear train.

Optionally, the cam follower is coupled to the shaft and rotates in conjunction with the shaft. Optionally, the cam follower projects from a rotatable hub. Optionally, the cam includes at least one ramp portion and at least one step portion, and the impactor translates in a distal direction when the cam follower moves past the at least one step portion from the at least one ramp. Optionally, the cam includes multiple ramp portions and multiple step portions. Optionally, movement of the cam follower along the ramp portion causes an increase in biasing force of a biasing member. Optionally, the biasing member is a coil spring.

Optionally, the actuator is configured to translate the impactor up to 0.75 millimeters. Optionally, the actuator is configured to translate the impactor at least 0.25 millimeters. Optionally, a frequency of oscillatory translation of the impactor is up to 400 Hz. Optionally, a frequency of oscillatory translation of the impactor is at least 50 Hz. Optionally, the distal tip of the impactor includes one or more impact force focusing features.

Optionally, the elongate body and the impactor include guide wire lumens configured to receive a guide wire.

Optionally, the catheter includes a handle, wherein a motor is housed within the handle for rotating the shaft. Optionally, the motor is battery powered.

According to an aspect, a method of treating an occlusion in a body lumen includes positioning a distal end of a catheter adjacent to the occlusion in the body lumen, wherein the distal end of the catheter includes an impactor configured to translate relative to an elongate body for impacting an occlusion in the body lumen, operating a motor to generate relative rotation and translation between a cam and a cam follower of the catheter, wherein the relative rotation and translation between the cam and the cam follower causes the impactor to translate in conjunction with the cam or the cam follower to impact the occlusion in the body lumen.

Optionally, the method includes inflating an enclosure of the catheter. Optionally, the enclosure is inflated prior to operating the motor to stabilize the distal end of the catheter within the body lumen. Optionally, the impactor is coupled to the cam and translates in conjunction with the cam. Optionally, the catheter includes a shaft extending within the elongate body that is rotated by the motor. Optionally, the shaft is coupled to the motor via a gear train. Optionally, the cam follower is coupled to the shaft and rotates in conjunction with the shaft.

Optionally, the cam includes a contact surface, the cam follower includes a rotatable hub and a projection extending from the hub and configured to move along the contact surface, and operating the motor to generate relative rotation between a cam and a cam follower causes the projection to move along the contact surface. Optionally, the contact surface includes at least one ramp portion and at least one step portion, and the impactor is configured to translate in a distal direction when the projection moves past the at least one step portion.

Optionally, the impactor translates up to 0.75 millimeters. Optionally, the impactor translates at least 0.25 millimeters. Optionally, the cam is coupled to the impactor and translates in conjunction with the impactor. Optionally, a frequency of the oscillatory translation is up to 400 Hz. Optionally, a frequency of the oscillatory translation is at least 50 Hz.

Optionally, the elongate body includes a guide wire lumen configured to receive a guide wire, and positioning the distal end of the catheter adjacent to the occlusion in the body lumen includes introducing a guide wire into the body lumen, and advancing the distal end of the catheter into the body lumen over the guide wire. Optionally, positioning the distal end of the catheter adjacent to the occlusion includes advancing the distal end of the catheter distally in the body lumen until the distal end contacts the occlusion.

Optionally, the occlusion includes fibrotic tissue and a distal end of the impactor includes at least one force focusing feature that includes a sloping portion that leads to a pointed portion. Optionally, the occlusion includes calcific tissue and a distal end of the impactor includes a plurality of spaced-apart regions.

According to an aspect, a catheter for treating an occlusion in a body lumen includes a catheter including an elongate body, a shaft disposed within the elongate body, an impactor forming a distal end of the catheter and configured to translate relative to the elongate body for impacting an occlusion in the body lumen, and an actuator configured to translate the impactor. According to an aspect, the catheter for treating an occlusion in a body lumen further includes a handle located at a proximal end of the catheter, a motor located in the handle and connected to the actuator, and a battery located in the handle for powering the motor.

### DESCRIPTION OF THE FIGURES

The invention will now be described, by way of example only, with reference to the accompanying drawings, in which:

FIG. 1A illustrates a perspective view of an exemplary catheter system, according to one or more examples of the present disclosure.

FIG. 1B illustrates a view of the exemplary catheter system of FIG. 1B positioned in a body lumen of a patient, according to one or more examples of the present disclosure.

FIGS. 1C and 1D illustrate aspects of an exemplary retractable sheath, according to one or more examples of the present disclosure.

FIG. 2 illustrates a perspective view of the interior of a proximal handle of a catheter, according to one or more examples of the present disclosure.

FIG. 3A illustrates a side cross-sectional view of a distal portion of an exemplary catheter, according to one or more examples of the present disclosure.

FIG. 3B illustrates a perspective view of a distal portion of an exemplary catheter device with an enclosure of the catheter removed, according to one or more examples of the present disclosure.

FIG. 3C illustrates a perspective view of a cam assembly of an exemplary catheter in a first position, according to one or more examples of the present disclosure.

FIG. 3D illustrates a perspective view of a cam assembly of an exemplary catheter in a second position, according to one or more examples of the present disclosure.

FIG. 3E illustrates a side view of a distal portion of an exemplary catheter showing a biasing member of the catheter in a compressed state, according to one or more examples of the present disclosure.

FIG. 3F illustrates a side view of a distal portion of an exemplary catheter showing the biasing member of the catheter in an extended state, according to one or more examples of the present disclosure.

FIG. 4A illustrates a first example of an impact force focusing feature that can be included at the distal end of an impactor of an exemplary catheter, according to one or more examples of the present disclosure.

FIG. 4B illustrates a second example of an impact force focusing feature that can be included at the distal end of an impactor of an exemplary catheter, according to one or more examples of the present disclosure.

FIG. 5 illustrates a cross-sectional view of an elongate body of a catheter showing various internal lumens of the elongate body, according to one or more examples of the present disclosure.

FIG. 6 illustrates a flowchart of an exemplary method of treating an occlusion in a body lumen using a catheter, according to one or more examples of the present disclosure.

### DETAILED DESCRIPTION

The following description is presented to enable a person of ordinary skill in the art to make and use the various embodiments and aspects thereof disclosed herein. Descriptions of specific devices, assemblies, techniques, and applications are provided only as examples. Various modifications to the examples described herein will be readily apparent to those of ordinary skill in the art, and the general principles described herein may be applied to other examples and applications without departing from the spirit and scope of the various embodiments and aspects thereof. Thus, the various embodiments and aspects thereof are not intended to be limited to the examples described herein and shown but are to be accorded the scope consistent with the claims.

Described herein are examples of lesion crossing catheters that can deliver direct mechanical impact forces to occlusions. The catheters include an impactor at their distal ends that is translatable in an oscillatory manner to deliver direct mechanical impact forces to occlusions in body lumens. During a treatment with a catheter, a distal portion of the catheter can be advanced through a body lumen to a region of the body lumen that is adjacent to an occlusion, such as a fibrotic or calcified occlusion, until the impactor at the distal end of the catheter is proximate the occlusion. Once the impactor is positioned proximate the occlusion, a motor of the catheter can be activated. The motor is operably coupled to an actuator that is configured to axially translate the impactor in an oscillatory manner to deliver mechanical impact forces to the occlusion. Oscillatory operation of the impactor may apply vibratory mechanical forces to the occlusion. As the impactor disrupts the occlusion, the catheter can be advanced further into the occlusion. When the catheter is used with a guide wire, the guide wire may move and oscillate in conjunction with the impactor to deliver additional mechanical forces to the occlusion.

Advantageously, the delivery of direct mechanical impact forces by the catheter may make the treatment of occlusions quicker and more effective than conventional catheter-based methods for treating occlusions in body lumens. Direct mechanical forces may beneficially allow a user to apply forces to a focused region of a lesion. The impactor can concentrate occlusion-clearing mechanical forces in a relatively small surface area, allowing the catheter to easily penetrate and clear treatment-resistant lesions such as calcified and fibrotic occlusions and CTOs. The impactor directs the mechanical forces in a forward (i.e., distal) direction, making the catheter well-suited for treating large occlusions, such as CTOs, that have substantially blocked a body lumen. Further, the dynamic mode of action of the catheter during treatment, whereby the impactor oscillates forward and backward within the body lumen, may allows a user to continuously penetrate and drill into occlusions in body lumens, streamlining the treatment of occlusions that extend longitudinally through a length of a body lesions.

In some embodiments, the lesion crossing catheter is a so-called “rapid exchange-type” (“Rx”) catheter provided with an opening through which a guide wire is guided (e.g., through a middle portion of a central shaft of the catheter in a longitudinal direction). In other embodiments, the catheter may be an “over-the-wire-type” (“OTW”) catheter in which a guide wire lumen is formed throughout the overall length of the catheter, and a guide wire is guided through an opening or guide wire port included in a proximal-end handle or hub of the catheter.

In the following description of the various embodiments, reference is made to the accompanying drawings, in which are shown, by way of illustration, specific embodiments that can be practiced. It is to be understood that other embodiments and examples can be practiced, and changes can be made without departing from the scope of the disclosure.

In addition, it is also to be understood that the singular forms “a,” “an,” and “the” used in the following description are intended to include the plural forms as well, unless the context clearly indicates otherwise. It is also to be understood that the term “and/or” as used herein refers to and encompasses any and all possible combinations of one or more of the associated listed items. It is further to be understood that the terms “includes,” “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof. As provided herein, it should be appreciated that any disclosure of a numerical range describing dimensions or measurements such as thicknesses, length, weight, time, frequency, temperature, voltage, current, angle, etc. is inclusive of any numerical increment or gradient within the ranges set forth relative to the given dimension or measurement.

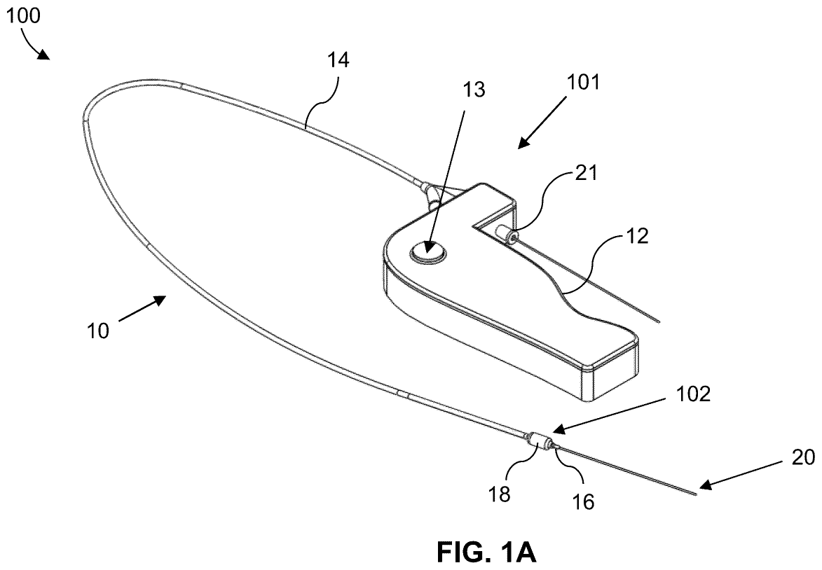

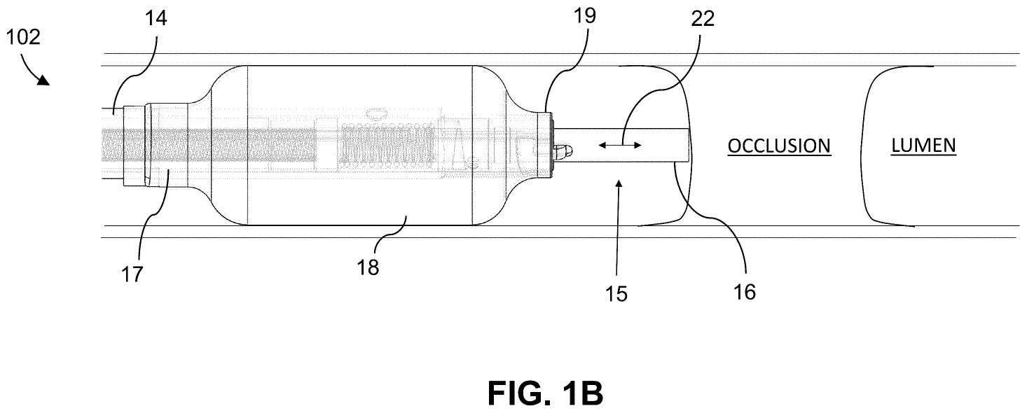

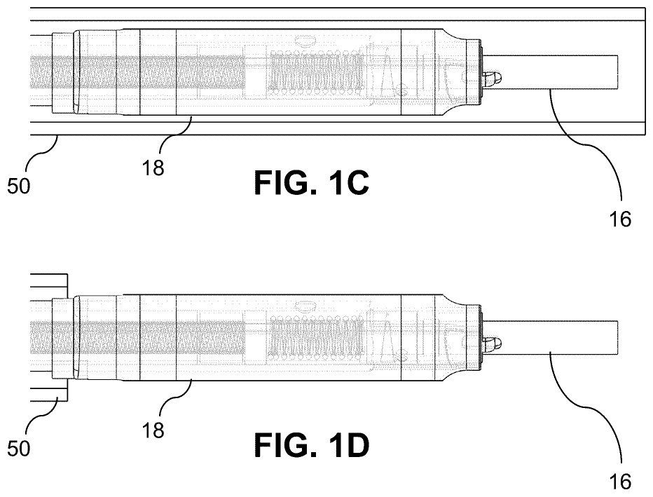

FIGS. 1A-1B depict an exemplary catheter system 100 for treating lesions in a body lumen, such as CTOs in a blood vessel wall or kidney stones in a ureter. As shown in FIG. 1A, the catheter system 100 includes a catheter 10 and, optionally, a guide wire 20. In some examples, a proximal portion 101 of the catheter 10 includes a handle 12. The handle 12 of the catheter 10 may house a power source and/or a motor for driving an impactor 16, as discussed in detail below. In some examples, the handle 12 includes a guide wire port 21 for inserting the guide wire 20 to facilitate insertion and positioning of the catheter 10 within the body lumen.

The catheter 10 includes an elongate body 14 that extends between the proximal portion 101 of the catheter and the distal portion 102 of the catheter. The elongate body 14 may be attached to the handle 12 at its proximal end and may extend from the handle to terminate at a distal portion 102 of the catheter 10. The elongate body 14 may extend through at least a portion of the length of an enclosure 18 included in the distal portion 102 of the catheter. In some examples, the elongate body 14 is formed from a flexible material that permits the maneuvering and torquing of the elongate body during navigation of the catheter 10 through a body lumen. For instance, the elongate body 14 may be formed from a compliant polymeric material. In some examples, the elongate body 14 includes various lumens and/or channels configured for carrying fluid, the guide wire 20, and other components of the catheter system 100 between the handle 12 in the proximal portion 101 of the catheter 10 and a distal portion 102 of the catheter, such as one or more lumens for carrying fluid to inflate and deflate the enclosure 18 and one or more lumens for aspirating debris formed when an occlusion is treated with the impactor 16.

FIG. 1B illustrates an example of the distal portion 102 of the catheter 10 positioned in a body lumen of a patient, such as a region of the patient's vasculature that includes one or more occlusions. As mentioned above, the distal portion 102 of the catheter 10 includes an impactor 16, which can be driven by a motor in the handle 12 to translate in a distal-proximal (longitudinal) direction 22 in an oscillatory manner to deliver mechanical forces to occlusions. The impactor 16 may be positioned distal to the elongate body 14 and translationally coupled to the elongate body, such that the impactor 16 can translate and oscillate relative to the elongate body 14 during a treatment with the catheter 10. In some examples, the impactor 16 is shaped as a longitudinal tube. However, in alternative examples the impactor 16 may be a different shape, such as a rod, a closed cylindrical shape, a frustoconical shape, a tapered shape, or some other shape. In some examples, a diameter of the impactor 16 is less than a diameter of the elongate body 14 of the catheter 10. Optionally, at least a portion of the impactor 16 extends within the elongate body 14. In some examples, the impactor 16 is formed from a rigid material, such as a metal (e.g., stainless steel) or a rigid polymeric material, which may improve the delivery of mechanical forces to occlusions. In some examples, the impactor 16 may be formed from a flexible material. In some examples, the impactor 16 is formed from two or more different materials, such as a relatively more rigid material in a proximal portion of the impactor and a relatively more flexible material at a distal end of the impactor (or vice versa).

The amount of translation of the impactor 16 may be selected to optimize the treatment of occlusions and reduce the risk of harm to healthy tissue in the body lumen. In some examples, the impactor 16 is configured to translate by approximately 0.5 mm (one half millimeter) in the distal-proximal direction 22 during operation of the catheter 10 (i.e., during oscillatory translation of the impactor responsive to operation of a motor of the catheter). In some examples the impactor 16 is configured to translate at least 0.25 mm (one quarter millimeter), at least 0.5 mm (one half millimeter), or at least 0.75 mm (three quarters of a millimeter) in the distal-proximal direction 22 during operation of the catheter 10. In some examples, the impactor 16 is configured to translate no more than 0.5 mm (one half millimeter), no more than 0.75 mm (three quarters of a millimeter), or no more than 1 mm (one millimeter) in the distal-proximal direction 22 during operation of the catheter 10. In some examples, the impactor 16 is configured to translate between 0.25 mm (one quarter millimeter) and 0.75 mm (three quarters of a millimeter) in the distal-proximal direction 22 during operation of the catheter 10. However, in other examples, the impactor 16 may be configured to translate a greater or lesser amount.

The frequency of translation of the impactor 16 (i.e., the oscillation frequency) may also be selected to improve treatment with the catheter 10. For instance, in some examples, the impactor 16 is configured to translate at an oscillation frequency of at least 50 Hz (fifty hertz), at least 100 Hz (one hundred hertz), at least 200 Hz (two hundred hertz), or at least 400 Hz (four hundred hertz). In some examples, the impactor 16 is configured to translate at an oscillation frequency of no greater than 50 Hz (fifty hertz), no greater than 100 Hz (one hundred hertz), no greater than 200 Hz (two hundred hertz), or no greater than 400 Hz (four hundred hertz). In some examples, the impactor 16 is configured to translate at an oscillation frequency between 50 Hz (fifty hertz) and 400 Hz (four hundred hertz). However, greater or lesser oscillation frequencies may be selected for particular applications.

As shown in FIG. 1B, the impactor 16 may extend more distally of an enclosure 18 of the catheter, such that a distal end 15 of the impactor is external to the enclosure and can directly contact occlusions in the body lumen during a treatment with the catheter 10. In some examples, the length of the distal end 15 of the impactor 16 is the range from two millimeters to twenty millimeters, preferably in the range from three millimeters to 10 millimeters. In some examples, the length of the distal end 15 of the impactor 16 is no less than 5 mm. In some examples, the length of the distal end 15 of the impactor 16 is no greater than 5 mm. In some examples, the length of the distal end 15 of the impactor 16 is great enough to promote efficient treatment of occlusions, i.e., to provide a sufficient length to maneuver the impactor 16 into complex longitudinal occlusions without requiring repeated repositioning of the catheter 10. The length of the distal end 15 of the impactor 16 may be small enough that the impactor does not risk damaging healthy tissue in the body lumen wall. In some examples, a proximal end of the impactor 16 is surrounded by the enclosure 18 and/or the elongate body 14 of the catheter. In some embodiments, the impactor 16 is retractable within the enclosure 18 to avoid damaging tissue when advancing the catheter to the treatment site. In some embodiments, a retractable sheath may be extended to surround the impactor 16 when advancing the catheter 10 to the treatment site and then retracted at or near the treatment site to expose the impactor 16. FIG. 1C illustrates an example of catheter 10 that includes a retractable sheath 50 in an extended position in which the retractable sheath 50 covers the impactor 16 (e.g., extends distally beyond a distal end of the impactor 16). FIG. 1D illustrates the retractable sheath 50 in a retracted position in which the impactor 16 is uncovered for treatment of an occlusion. The retractable sheath 50 may be retracted to uncover the enclosure 18 to enable the enclosure 18 to be expanded to contact the lumen wall. The retractable sheath 50 may be integrated into the catheter 10 or may be a separate component, such as an introducer that is advanced to the treatment site with the catheter retained within.

As mentioned above, the distal portion 102 of the catheter 10 includes the enclosure 18, which extends circumferentially around and surrounds at least a portion of the elongate body 14. In some examples, the enclosure 18 is formed from a compliant or semi-compliant material. An example of a suitable material is an elastomeric polymer. In some examples, the enclosure 18 is a balloon, such as an inflatable angioplasty balloon. In some examples, a proximal end 17 and a distal end 19 of the enclosure 18 are attached to regions of the distal portion of the elongate body 14 (e.g., via heat sealing or an adhesive). Accordingly, the enclosure 18 may form a sealed volume around at least a portion of the elongate body 14 and/or a portion of the impactor 16. When the catheter 10 is positioned in a body lumen and prior to initiating treatment with the catheter, the enclosure 18 may be filled with a fluid. In some examples, the fluid is saline or another fluid compatible with (e.g., isotonic with) body fluid in the body lumen. When filled with the fluid, the enclosure 18 may expand to contact the walls of the body lumen and provide an annular channel around the elongate body 14. Advantageously, the inflated enclosure 18 may also function as a centering mechanism within the body lumen. For instance, when the enclosure 18 is inflated with fluid, the impactor 16 of the catheter may be maintained at approximately the center of the body lumen, such that there is a reduced risk of harming healthy tissue in the walls of the body lumen during oscillatory translation of the impactor 16. In some examples, the enclosure 18 is in a non-expanded state, and optionally in a folded state, during positioning of the catheter 10 within a body lumen. In a non-expanded or folded state, the crossing profile of the enclosure 18 is reduced to improve the maneuverability of the catheter 10 through the body lumen.

During a treatment with the catheter system 100, a physician may position the catheter 10 within a body lumen by inserting the guide wire 20 into the body lumen (e.g., an occluded blood vessel, valve, or ureter) and then advancing the catheter over the guide wire until the distal end 15 of the impactor 16 is positioned adjacent to an occlusion targeted for treatment, as illustrated in FIG. 1B. The physician may advance the catheter 10 until the physician perceives resistance resulting from contact of the distal end 15 of the impactor 16 with the occlusion, which may indicate to the physician that the occlusion has been reached. The physician may leave the distal end 15 of the impactor 16 in contact with the occlusion or may move the catheter 10 back proximally to create a small amount of space between the distal end 15 of the impactor 16 and the occlusion. Additionally, or alternatively, the physician may track the position of the guide wire 20 and catheter 10 within the body lumen using real-time and/or static imaging devices, including x-ray imaging, intravascular ultrasound (IVUS), optical coherence tomography (OCT), radiofrequency (RF) navigation, and other such techniques. Once the distal end of the catheter 10 has been positioned adjacent to the occlusion (in contact with or spaced apart from) in the body lumen, the enclosure 18 may be inflated with fluid such that the outer walls of the enclosure contact the walls of the body lumen. Inflation of the enclosure in the body lumen may stabilize the distal end of the catheter within the body lumen so that the impactor remains substantially centered within the body lumen and/or may anchor the impactor 16 to the body lumen. Stabilizing within and/or anchoring to the body lumen may facilitate treatment of occlusions by enhancing impact to the occlusions and/or may reduce the risk of harm to healthy tissue of the lumen walls. Optionally, the enclosure 18 is not inflated at this stage of the process. A user of the catheter system 100 may then initiate treatment with the catheter 10 by activating the oscillatory translation of the impactor 16 in a distal-proximal direction 22.

In some examples, a power source of the catheter 10 is activated via a user input, such as the press of a power switch 13 included on the handle 12 shown in FIG. 1A. In some examples, the power switch 13 is a dial or another user interface that allows a user to control various aspects of the treatment, such as the oscillation frequency of the impactor 16. In such examples, the user may select an oscillation frequency of the impactor via a user input on the power switch 13 or by selecting the oscillation frequency using a dial or another user interface of the catheter. The user may apply a distally directed force to the elongate body 14 to assist the impactor 16 in delivering repeated mechanical forces directly to the occlusion. The repeated mechanical forces delivered via oscillatory translation of the impactor 16 may then be used to disrupt the occlusion. Continuous distal force applied by the user may enable the impactor 16 to move further into the occlusion as it breaks up occlusive material. Optionally, the user may cease activation of the impactor 16 to adjust the position of the catheter 10 within the body lumen and resume an additional stage of treatment with the catheter. Once the occlusion has been adequately treated with the catheter 10 (e.g., when the catheter is able to cross the area of the occlusion), the user may optionally inflate the enclosure 18 further (e.g., to a pressure greater than 5 atm) to enlarge the lumen. The user may then continue treatment with the catheter 10. When the body lumen has been sufficiently treated, the enclosure 18 may be inflated further or deflated and the catheter 10 may be removed. Additionally, or alternatively, the user may continue treatment with a different catheter device, such as any of the catheter devices described and incorporated by reference herein. In some examples, if the catheter 10 is disposable, the physician may discard the catheter following treatment. In some examples, the handle 12 of the catheter 10 is reusable and various components in a distal portion 102 of the catheter (e.g., the elongate body 14, impactor 16, enclosure 18, and actuator) may be discarded following treatment.

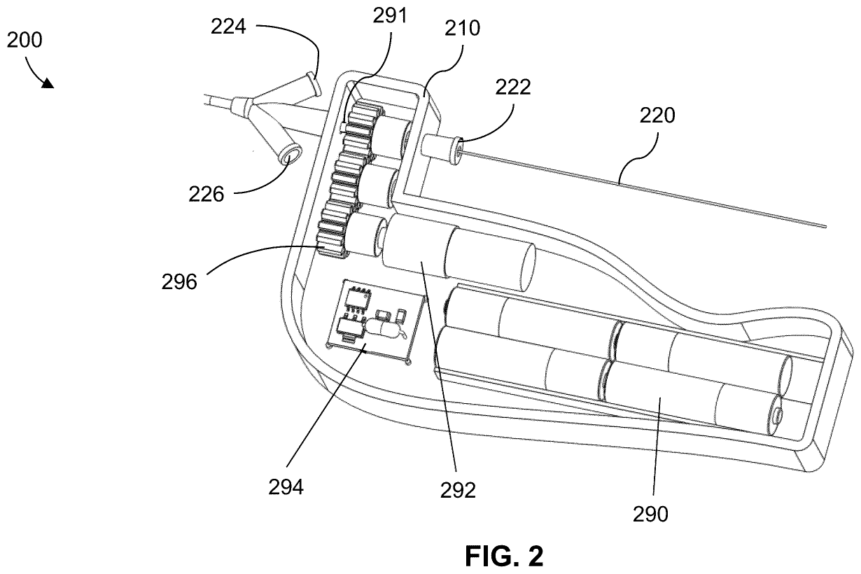

The handle 12 of the catheter 10 may house various components at the proximal end of the catheter for operating the impactor, such as a power source, a motor, a control circuit, and a gear train. FIG. 2 illustrates the interior of a handle 200 of an exemplary catheter. Handle 200 can be used for handle 12 of catheter 10 of FIGS. 1A-1B. The handle 200 includes a power source 290, a motor 292, a control circuit 294, and a gear train 296, each housed within a housing 210, which may be formed from a rigid polymeric material or a metal.

The power source 290 may provide power to the motor 292 (e.g., a DC motor) to operate the motor to drive oscillatory translation of the impactor at the distal end of the catheter. In some examples, the motor 292 is a DC motor configured to drive a flexible shaft 291 of the catheter. In some examples, a gear train 296 is operationally coupled to the motor 292 and the shaft 291 to reduce the rotational speed and increase the torque applied to the shaft 291.

In some examples, the power source 290 of the catheter includes one or more batteries, such as one or more commercially available lithium-ion, lead-acid, nickel-cadmium, and/or alkaline batteries. In some examples, one or more of the batteries includes a rechargeable battery. The one or more batteries may be arranged in a battery pack. In some examples, the speed, frequency, magnitude, power, or duration of the treatment (e.g., the speed, frequency, magnitude, power, or duration of the actuation of the catheter's impactor) is based on the number of battery cells included in the power source 290. For instance, a power source 290 with a greater number of battery cells may be capable of providing an increased amount of power to the motor 292 during operation, and may therefore permit actuation of the impactor at an increased speed, oscillation frequency, magnitude, or power. In a particular example, a 4-cell battery pack may power the motor 292 to operate at approximately 6,000 RPM (six thousand rotations per minute) or more, and/or may allow for actuation of the impactor at an oscillation frequency of approximately 100 Hz (one hundred hertz). However, the power source 290 may include a greater or fewer number of cells or batteries and may be configured to provide more or less power to the motor 292. In various examples, power source 290 may be configured to provide power sufficient to translate the impactor at an oscillation frequency of at least 50 Hz (fifty hertz), at least 100 Hz (one hundred hertz), at least 200 Hz (two hundred hertz), or at least 400 Hz (four hundred hertz).

Advantageously, using batteries as a power source 290 allows the catheter to be used in environments where other power sources (e.g., AC power provided by electrical outlets or an external power generator required by other devices) may be unavailable or cumbersome to use. The use of batteries as a power source 290 may also make the catheter cheaper to manufacture and use, such that the catheter may be a single-use or disposable catheter that is designed to be discarded after a single treatment or series of treatments. However, in some examples, the catheter is wired to an external power source. In some examples, the catheter may be selectively powered by either battery power or by wired connection to an external power source. In some examples, the power source 290 includes rechargeable batteries and a connector for connecting a charging cord.

The control circuit 294 of the catheter may be configured to control the operation of the motor 292 during a treatment with the catheter. For instance, the control circuit 294 may selectively supply power from the power source 290 to the motor 292 to initiate operation of the motor and oscillation of the catheter's impactor and/or terminate operation of the motor and oscillation of the impactor. In some examples, the control circuit 294 is configured to initiate operation of the motor 292 responsive to a user input on a power switch included in the handle 200 (e.g., a press of the power switch 13 shown in FIG. 1). In some examples, the control circuit 294 is configured to terminate operation of the motor 292 responsive to termination of the user input on the power switch (e.g., the user ceasing the pressing of the power switch or moving a switch from a first position to a second position). In some examples, the control circuit 294 is a self-terminating control circuit configured to automatically terminate operation of the motor 292. For instance, the control circuit 294 may be configured to automatically terminate operation of the motor 292 after a certain period of time, which may correspond to the amount of time required for a typical treatment with the catheter. In some examples, the control circuit 294 may be configured to automatically terminate operation of the motor 292 responsive to a determination that the power source 290 has below a threshold amount of power (e.g., one or more batteries are nearly depleted), or that the power source has no power left to continue treatment. Advantageously, the control circuit 294 may ensure that a uniform amount of power is provided to the motor 292 during a treatment to cause more uniform oscillatory translation of the impactor to break up occlusions. In examples where the catheter is disposable, a self-terminating control circuit 294 may advantageously reinforce the disposability of the catheter (i.e., by preventing a user from operating the catheter after a certain amount of time or after a certain number of treatments). In some examples, the control circuit 294 may be used to adjust an oscillation frequency of an impactor of the catheter (e.g., the impactor 16). For instance, a user may control the oscillation frequency of the impactor using a user interface of the catheter (e.g., a dial or the power switch 13), and the control circuit may operate the motor at a speed that corresponds to a user input on the user interface.

The handle 200 of the catheter 10 may include various ports that provide openings into one or more lumens included in the elongate body 14 of the catheter. For instance, the handle 200 may include a flush port 224 configured to introduce fluid into the body lumen during a treatment, e.g., to flush debris created in the body lumen during treatment of an occlusion with the impactor 16. The flush port 224 may be in fluid communication with a flush lumen in the elongate body of the catheter. In some examples, the catheter 10 further includes a guide wire port 222 configured to receive a guide wire 220 into a guide wire lumen of the elongate body. The guide wire port 222 may be sized to receive a commercially available guide wire 220 used for peripheral or coronary applications, such as a guide wire having a diameter of approximately 0.014″ (fourteen thousandths of an inch). In some examples, the guide wire port 222 may also be used as an aspiration port. In such examples, fluid and/or debris may be evacuated from the body lumen via a guide wire lumen of the catheter and through the guide wire port 222. However, in some examples, the catheter 10 includes a separate guide wire port and aspiration port in fluid communication with a respective guide wire lumen and aspiration lumen. In some examples, fluid may be continuously flowed through the treatment area near the distal end of the catheter during a treatment to remove debris generated by the treatment of occlusions with the impactor. In such examples, fluid may be continuously flowed into the body lumen via the flush port 224 (and via a flush lumen) and fluid and debris may be continuously flowed out of the body lumen via the aspiration port (e.g., guide wire port 222 or a separate aspiration port) (and via an aspiration lumen). In some examples, the catheter 10 includes an inflation port 226 for flowing fluid from the proximal end of the catheter into the enclosure. The inflation port 226 may be in fluid communication with an inflation lumen in the elongate body of the catheter. During a treatment with the catheter, fluid may be flowed into the inflation port 226 and through the inflation lumen to inflate and/or deflate the enclosure.

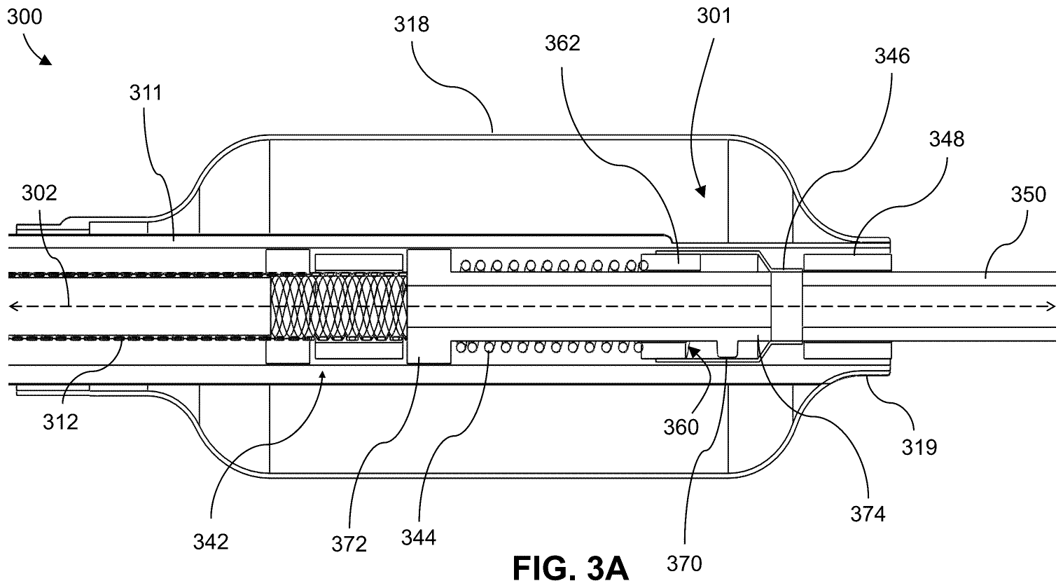

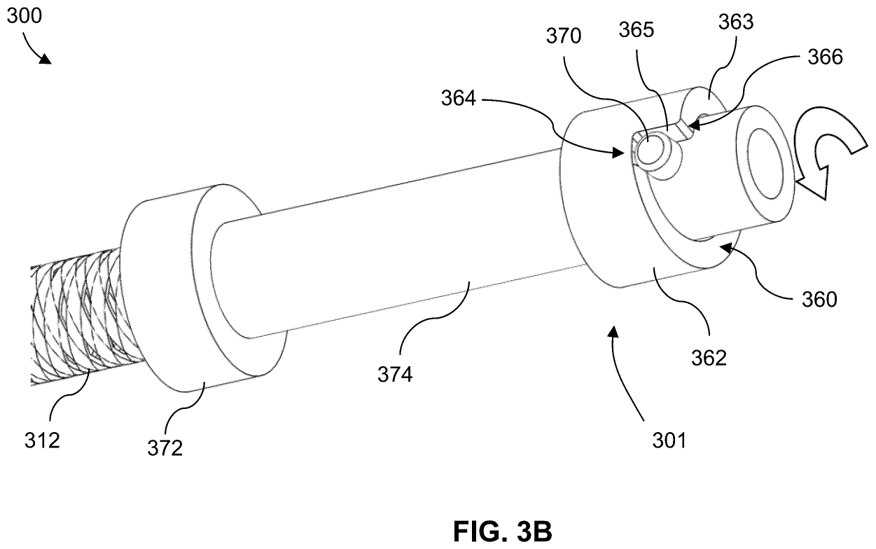

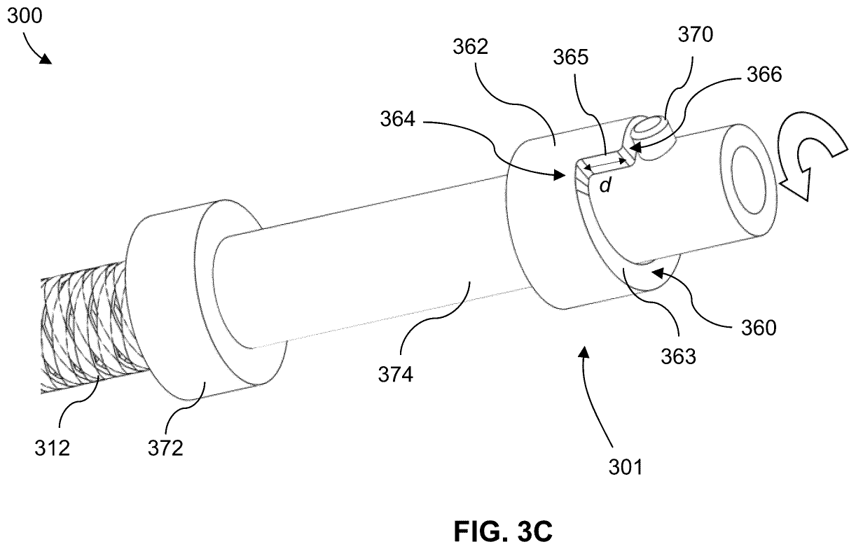

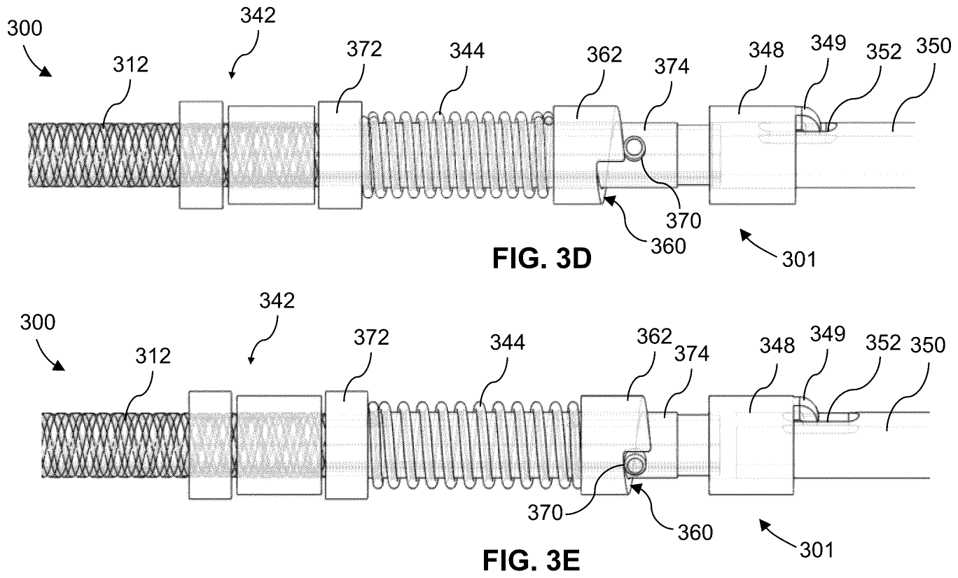

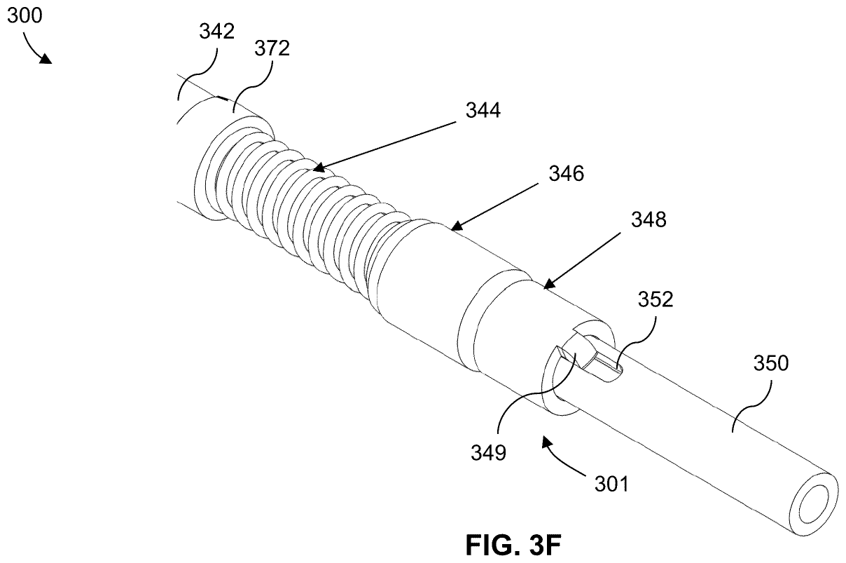

FIGS. 3A-3F depict a distal portion 300 of an exemplary catheter, such as the catheter 10 shown in FIGS. 1A-1B. As noted above, a distal portion 300 of the catheter is inserted into the body lumen of a patient and positioned adjacent to an occlusion targeted for treatment by the catheter. Accordingly, the distal portion 300 of the catheter may include one or more elements of the catheter that are used for positioning the catheter in the body lumen and delivering the oscillatory forces to the occlusions, such as an enclosure 318, an impactor 350, an actuator 301 for translating the impactor responsive to operation of the motor, and a shaft 312 for operationally coupling the actuator 301 with the motor in the handle. As mentioned above, various components of the distal portion 300 of the catheter, such as the actuator 301, may be enclosed by the enclosure 318, which surrounds and forms a sealed volume around the elongate body 311. In some examples, a distal end 319 of the enclosure 318 is sealed to a region of the elongate body 311 near the catheter's distal end. In some examples, the distal end 319 of the enclosure 218 is conterminous with the elongate body 311.

In some examples, the shaft 312 extends along the elongate body 311 between a proximal portion of the catheter (e.g., the handle 200 shown in FIG. 2) and the actuator 301 in the distal portion 300 of the catheter. The shaft 312 may be formed from a flexible material that permits the bending and torquing of the shaft 312 during navigation and positioning of the catheter in a body lumen. In some examples, the shaft 312 includes a wire mesh tubing, optionally embedded within a polymeric tube. In a particular example, the shaft 312 includes a stainless-steel mesh embedded within a polymeric tube. Advantageously, the shaft 312 may be formed from a resilient material that prevents or minimizes circumferential twisting or deformation of the shaft during rotation. In some examples, a friction-reducing element (e.g., a coating or fluid) may be included between the elongate body 311 and the shaft 312 to prevent friction between the shaft and the elongate body during rotation of the shaft 312 relative to the elongate body 311.

In some examples, the shaft 312 is operably coupled at its proximal end to the motor (e.g., via a gear train as shown in FIG. 2) and is coupled at its distal end to one or more components of the actuator 301. When the motor is operated, the shaft 312 is configured to rotate, causing one or more components of the actuator 301 to rotate to drive the oscillatory translation of the impactor 350. In the illustrated example, the shaft 312 is connected at its distal end to a rotatable hub 374, such that a rotation of the shaft 312 results in rotation of the rotatable hub 374. For example, the shaft 312 may be welded or press fitted together with the rotatable hub 374. The rotatable hub 374 carries a cam follower 370 such that the cam follower 370 revolves around a longitudinal axis 302 of the rotatable hub 374 with rotation of the rotatable hub 374. The cam follower 370 “follows” (e.g., rolls or slides along) a cam 360 of a cam body 362. The cam body 362 is biased in the distal direction so that the cam follower 370 and cam 360 are in continuous engagement. The cam body 362 is biased by a biasing member 344 (e.g., a coil spring), the proximal end of which abuts a flange 372 of the rotatable hub 374, and the distal end of which abuts the cam body 362. One or more thrust bearings 342 maintain the axial position of the rotatable hub 374, while allowing the rotatable hub 374 to freely rotate.

As the cam follower 370 revolves around the longitudinal axis 302 of the rotatable hub 374 as the rotatable hub 374 rotates, the cam body 362 axially translates according to the shape of the cam 360. The impactor 350 may be coupled to the cam body 362 via a cam-tip coupler 346 such that the axial translation of the cam body 362 results in axial translation of the impactor. An anti-rotational feature 348 may be fixedly mounted to the distal end of the elongate body 311 and may include a tab 349 that extends into a slot 352 of the impactor 350, as shown best in FIGS. 3D and 3E. The positioning of the stationary tab 349 in the slot 352 prevents the impactor 350 (and the components coupled thereto) from rotating, while allowing the impactor to translate.

FIGS. 3B-3C illustrate the relative rotation and translation of the cam 360 and the cam follower 370 of the actuator 301 to cause the oscillatory translation of the impactor 350 during a treatment with the catheter. In some examples, the cam 360 includes at least one ramp portion 363 and at least one step portion 365. The ramp portion 363 may slope around at least a portion of the circumference of the cam 360. The cam 360 may have a uniform slope across the ramp portion 363 of the cam 360 or a varying slope. The transition between the most distal region 366 of the ramp portion 363 and the most proximal region 364 of the ramp portion 363 is referred to as a step portion 365. The step portion 365 is oriented at an approximately 90-degree angle relative to the ramp portion 363 of the cam 360.

As shown in FIGS. 3B-3C, the rotation of the rotatable hub 374 relative to the cam body 362 (e.g., in a counterclockwise direction as indicated by the arrows in FIGS. 3B-3C) may cause the cam follower 370 to move across the cam 360. In the first position shown in FIG. 3B, the cam follower 370 is positioned in contact with the most proximal region 364 of the ramp portion 363 of the cam 360. In the first position, the cam body 362 and the impactor 350 are distally translated relative to the rotatable hub 374. As the rotatable hub 374 rotates relative to the cam body 362, the cam follower 270 advances relatively gradually “up” the ramp portion 363, causing the cam body 362 to gradually translate in a proximal direction relative to the rotatable hub 374. As the rotatable hub 374 continues to rotate relative to the cam body 36, the cam follower 370 continues to move across and “climb” the ramp portion 363 of the cam 360 and the cam body 362 will continue to translate in a proximal direction until the cam follower 370 reaches the most distal region 366 of the ramp portion 363. In the second position shown in FIG. 3C, the cam follower 370 is positioned in contact with the most distal region 366 of the ramp portion 363 of the cam 360. In the second position, the cam body 362 and the impactor 350 may be proximally translated relative to the cam follower 370. To complete a full rotation of the rotatable hub 374 relative to the cam body 362, the cam follower 370 moves past the step portion 365 of the cam 360. When the cam follower 370 passes across the step portion 365, the cam body 362 accelerates quickly in a distal direction due to the force of the biasing member 344 until the cam follower 370 returns to contact the cam 360 at the most proximal region 364 of the ramp portion 363. The quick distal movement of the cam body 362 creates relatively high impact forces between the impactor 350 and an occlusion. Further rotation of the rotatable hub 374 relative to the cam body 362 will initiate another translation of the cam follower 370 around the cam 360. Repeated rotation of the rotatable hub 374 relative to the cam body 362 causes the impactor 350 to repeatedly translate axially in an oscillatory manner. The oscillatory translation of the impactor 350 may be primarily driven by energy stored in the biasing member 344 during rotation of the cam follower 370 across the ramp portion 363 and released as the cam follower 370 passes across the step portion 365.

The exemplary cam 360 shown in FIGS. 3A-3F includes one ramp portion 363 and one step portion 365. However, a cam 360 could include multiple ramp portions 363 and step portions 365 along its circumference (e.g., in a sawtooth pattern). For instance, the cam 360 could include two ramp portions separated by a respective two step portions, three ramp portions separated by a respective three step portions, four ramp portions separated by a respective four step portions, or a greater number of ramp portions and step portions. In such examples, one rotation of the rotatable hub 374 relative to the cam body 362 will cause impactor 350 to translate distally a number of times equal to the number of step portions around the circumference of the cam. For instance, in an example where the cam 360 includes three ramp portions and three respective step portions, relative rotation of the rotatable hub 374 relative to the cam body 362 will cause the impactor 350 to translate distally three times, i.e., one distal translation for each time the cam follower 370 moves past each of the step portions of the cam 360. Accordingly, the number of translations of the impactor 350 during each full rotation of the rotatable hub 374 relative to the cam body 362 may be based on the number of ramp portions 363 and step portions 365 included in the cam 360.

The depth of the step portion 365 (indicated by the distance “d” in FIG. 3C) may be approximately equal to the distance of translation of the impactor 350 during a full rotation of the cam follower 370 relative to the cam 360. In some examples, the step portion 365 of the cam 360 is approximately 0.75 mm (three-quarters of a millimeter), and the actuator 301 is configured to translate the impactor 350 up to 0.75 mm. However, as described elsewhere in the disclosure, catheters may be manufactured with impactors 350 configured to translate greater or lesser amounts, and the step portion 365 of the cam 360 may be designed with a depth corresponding to the desired translation distance of the impactor.

Accordingly, the depth of the step portion 365 may be selected to promote optimal treatment of occlusions during oscillatory translation of the impactor 350. For instance, a relatively small depth of the step portion 365 may provide for a smaller amount of distal-proximal translation of the impactor 350 when the cam follower 370 moves past the step portion 365. A larger depth of the step portion 365 may provide for a larger amount of distal-proximal translation of the impactor 350 when the cam follower 370 moves past the step portion 365. Likewise, the slope of the ramp portion 363 may be based at least partially on the depth of the step portion 365 of the cam 360. For instance, as the depth of the step portion 365 increases, the slope of the ramp portion 363 may be increased correspondingly. Advantageously, a ramp portion 365 with a more gradual slope may prolong the life of the catheter by reducing the amount of friction between the cam 360 and the cam follower 370 and decreasing the amount of power used during operation of the motor. Such advantages may be balanced with advantages provided by an increased depth of the step portion 365, such as increased axial translation of the impactor 350 and improved delivery of force to penetrate and cross occlusions in vasculature. Accordingly, catheters may be designed with cams 360 having a range of slopes of the ramp portion(s) 363 and depths of the step portion(s) 365 to provide catheters with improved performance for treating occlusions in body lumens.

As described above, the cam follower 370 includes a rotatable hub 374 that may be concentric with the cam 360. In some examples, the rotatable hub 374 fits within the cam body 362, with a tolerance to permit low-friction rotation of the rotatable hub 374 relative to the cam body 362. As mentioned above, the rotatable hub 374 may be coupled to the shaft 312 of the catheter such that the rotatable hub 374 rotates in conjunction with the shaft 312 when the motor is operated. In some examples, the rotatable hub 374 is configured to rotate within the inner circumference of the cam body 362. The cam follower 370 extends laterally from the rotatable hub 374 to contact the cam 360. The cam follower 370 may be configured to move along the cam 360 (i.e., slide or roll across a contact surface of the cam) during relative rotation of the rotatable hub 374 and cam body 362. In some examples, the cam follower 370 remains in contact with the cam 360 during relative rotation of the cam follower 370 and the cam 360. For instance, the force of the biasing member 344 may maintain contact between the cam 360 and the cam follower 370 during the relative rotation. In some examples, wherein the cam body 362 encircles the outer circumference of the rotatable hub 374, the cam follower 370 extends outwardly from the rotatable hub 374 to contact the cam 360. However, in other examples, the cam 360 encircles the inner circumference of the rotatable hub 374, and the cam follower 370 extends inwardly from the rotatable hub 374 to contact the cam 360.

The biasing member 344 may be arranged around the circumference of the shaft 312 of the catheter and/or a component of the actuator 301 (e.g., the rotatable hub 374). A distal end of the biasing member 344 may be in contact with one or more of the cam body 362 and the rotatable hub 374 and configured to apply a force to the cam body 263 and/or the rotatable hub 374. In some examples, the biasing member 344 is mechanically coupled to one or more of the cam body 362 and/or the rotatable hub 374. In a particular example, a distal end of the biasing member 344 is coupled to a proximal end of the cam body 362.

The biasing member 344 is configured to store potential energy when the length of the biasing member is compressed in an axial direction. The biasing member 344 may be further configured to release potential energy, e.g., when the length of the biasing member is expanded in an axial direction. In some examples, the oscillatory translation of the impactor 350 is powered by the potential energy of the biasing member 344. For instance, the release of potential energy from the biasing member 344 may cause the advancement of the impactor 350 in the distal direction. In some examples, the potential energy of the biasing member 344 is increased upon the return of the impactor 350 in a proximal direction. During relative rotation of the rotatable hub 374 and the cam body 362, the biasing member 344 may be configured to repeatedly store and then release potential energy to cause the impactor 350 to oscillate.

FIGS. 3D-3E illustrate the compression and expansion of a biasing member 344 during relative rotation of the rotatable hub 374 and cam body 362. FIG. 3D shows the actuator 301 in a first position. In the first position, the cam follower 370 is positioned at the proximal region 364 of a ramp portion 365 of the cam 360, and the cam body 362 and impactor 350 are translated in a proximal direction relative to the rotatable hub 374. In some examples, in the first position, the biasing member 344 is compressed in an axial direction and stores potential energy. FIG. 3E shows the actuator 301 in a second position. In the second position, the cam follower 370 is positioned at the distal region 366 of the ramp portion 365 of the cam 360 and the cam body 362 and impactor 350 are translated in a distal direction relative to the rotatable hub 374. In some examples, in the second position, the biasing member 344 is expanded in an axial direction and has released its stored potential energy. The release of the stored potential energy may drive the impactor 350 in the distal direction to deliver a direct mechanical force to an occlusion.

In some examples, one or more thrust bearings 342 are positioned proximal to the biasing member 344. In some examples, the thrust bearings 342 are configured to rotate in conjunction with (or in relation to) the rotation of the shaft 312. Advantageously, the thrust bearing 342 may improve the dynamic stability of the catheter during oscillatory translation of the impactor 350 by absorbing at least a portion of the axial load produced by the oscillatory translation. The thrust bearing 342 may be arranged around at least a portion of the circumference of the shaft 312 and optionally encircle the entire circumference of the shaft 312. The thrust bearing 342 may be mechanically coupled to the shaft 312, e.g., by way of an adhesive, such that the thrust bearing rotates in conjunction with the shaft 312. In some examples, the thrust bearing 342 may be positioned proximal to the cam-tip coupler 346, the cam 360, the cam follower 370, and/or the anti-rotational feature 348 of the actuator 301. In some examples, the catheter includes two or more thrust bearings arranged along the length of the shaft 312.

In some examples, mechanical coupling between the impactor 350 and the cam body 362 and/or rotatable hub 374 is provided by a cam-tip coupler 346. In a particular example, a proximal end of the cam-tip coupler 346 is coupled to the cam 360, and a distal end of the cam-tip coupler 346 is coupled to the impactor 350. However, in some examples the proximal end of the cam-tip coupler 346 is coupled to the rotatable hub 374. The coupling of the cam-tip coupler 346, the impactor 350, and the cam body 362 or rotatable hub 374 may be via heat-scaling, an adhesive, an interlocking fit, or some other means of mechanically fixing the elements of the actuator 301 and impactor 350. In some examples, at least a portion of the cam-tip coupler 346 encircles at least a portion of the circumference of the shaft 312, the cam body 362, and/or the rotatable hub 374. In some examples, at least a portion of the cam-tip coupler 346 encircles the entire circumference of the shaft 312, the cam body 362, and/or the rotatable hub 374. In some examples, the cam-tip coupler 346 surrounds and encloses the cam body 362 and/or the rotatable hub 374.

In some examples, the actuator 301 includes an anti-rotational feature 348 that remains rotationally stationary and is configured to prevent rotation of the impactor 350 during operation of the catheter. The anti-rotational feature 348 may be operationally engaged with at least the impactor 350 to prevent rotation of the impactor during operation of the motor and distal-proximal oscillation of the impactor. To ensure the anti-rotational feature 348 does not rotate during operation of the motor, the anti-rotational feature may be mechanically coupled to a stationary component of the catheter, such as an inner surface of the elongate body 311. Limiting the rotation of the impactor 350 may reduce the risk of harm to healthy tissue in a body lumen, e.g., by preventing unintended frictional motion between the impactor and the body lumen. In some examples, engagement of the anti-rotational feature 348 with the impactor 350 may additionally prevent the rotational movement of components mechanically coupled to the impactor, such as one or more of the cam body 362 and the cam-tip coupler 346.

The anti-rotational feature 348 can be engaged with the impactor 350 to prevent rotational movement of the impactor 350 while permitting oscillatory translation of the impactor in a distal-proximal direction. For instance, as shown in FIG. 3F, the anti-rotational feature 348 may feature one or more tabs 349 configured to engage with one or more slots 352 extending into the material of the impactor 350. In some examples, the tab 349 of the anti-rotational feature 348 extend into the slot 352 of the impactor 350 in the assembled catheter. The relative size of the tab 349 and slot 352 may prevent the rotational movement of the impactor 350 while the tab 349 of the anti-rotational feature 348 is engaged with the slot 352 of the impactor 350. For instance, the width of the slot 352 in a circumferential direction may be approximately equal to (or slightly greater than) the diameter of the tab 349, such that engagement of the tab with the slot 352 applies a mechanical force on an interior surface of the slot 352 to prevent the rotational movement of the impactor 350. The length of the slot 352 in the distal-proximal direction may be greater than the diameter of the tab 349, such that the impactor 350 can move freely for a distance in a distal or proximal direction unimpeded by the anti-rotational feature 348 (e.g., move freely for at least the distance of translation of the impactor 350 during operation of the catheter). In some examples, the anti-rotational feature 348 extends around at least a portion of the circumference of the impactor 350, and optionally extends around the entire circumference of the impactor. In such examples, the tab 349 may extend inwardly into the slot 352 of the impactor 350. In some examples, the anti-rotational feature 348 is coupled to the elongate body 311 such that the anti-rotational feature remains fixed relative to the elongate body, which may remain stationary during rotation of the shaft 312.

In some examples, one or more components of the actuator 301 may be formed from a metal, such as a stainless steel. In such examples, the mechanical coupling of various components of the actuator 301 and/or impactor 350 may be via welding, an adhesive, or an interlocking fit. Additionally, or alternatively, one or more components of the actuator 301 may be formed from a rigid polymer. In such examples, the mechanical coupling may be via heat sealing, an adhesive, or an interlocking fit. In some examples, the material of one or more components of the actuator 301 is the same as the material of the elongate body 311 and/or the shaft 312.

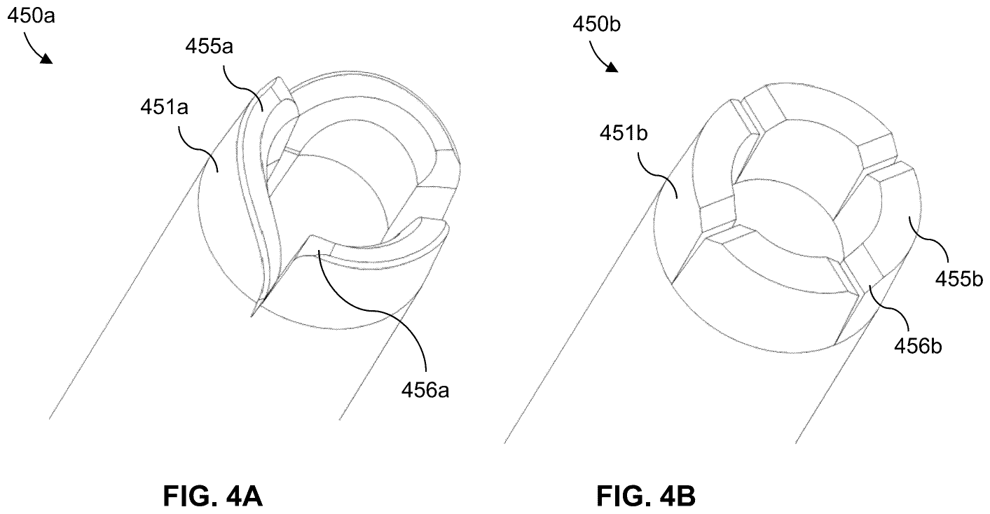

In some examples, the impactor 350 of the catheter includes one or more impact force focusing features designed to improve the delivery of mechanical forces to occlusions in a body lumen. For instance, a distal end of the impactor 350 may include the one or more impact force focusing features. The impact force focusing feature(s) may localize impact force on one or smaller regions of the occlusions relative to an impactor without the force focusing feature(s). In some examples and/or in some applications (e.g., when treating fibrotic tissue), the impact force focusing feature(s) may cut into the occlusion. FIGS. 4A-4B illustrate two exemplary impact force focusing features that may be included in an impactor 450a-450b of an exemplary catheter, such as the catheter 10 shown in FIG. 1. The impact force focusing feature(s) may advantageously improve the delivery of mechanical forces to the occlusion by reducing the surface area of the distal end 451a-451b of the impactor 450a-450b that contacts the occlusion when the impactor is advanced into the occlusion. By reducing the surface area of the distal end 451a-451b, the impactor 450a-450b may provide an increased amount of force to the occlusion compared to an impactor that lacks the impact force focusing features.

In some examples, the impact force focusing features are formed by modifying the distal end 451a-451b of the impactor to change the shape of the distal end to improve the delivery of the mechanical forces. For instance, the impact force focusing features may be formed by removing material from the distal end 451a-451b of the impactor 450a-450b (e.g., created by removing material after fabricating the impactor). Additionally, or alternatively, the impact force focusing features may be formed concurrently with formation of the impactor 450a-450b (e.g., may be created when molding, shaping, or forming the impactor from the material of the impactor). In such examples, the impact force focusing features may be formed from the same material as the impactor 450a-450b. In yet further examples, the impact force focusing features may include one or more elements that are joined to the impactor 450a-450b at or near its distal end 451a-451b. For instance, in some examples, impact force focusing features may be added to an impactor 450a-450b by coupling additional material forming the impact force focusing features to the distal end 451a-451b of the impactor, e.g., by heat sealing the impact force focusing features to the material of the impactor or by adhering the impact force focusing features to the material of the impactor using an adhesive. In such examples, the material of the impact force focusing features may be more rigid than the material of the impactor 450a-450b, e.g., to facilitate the penetration and tearing of occlusions. However, in other examples, the material of the impact force focusing features may be less rigid than the material of the impactor 450a-450b, e.g., in order to prevent damage to healthy tissue in the body lumen during oscillatory translation of the impactor.

As shown in FIG. 4A, in some examples, the impact force focusing features include thinned and/or sloping regions 455a. For example, each impact force focusing feature may include a sloping portion 455a that terminates in a distal pointed portion456a. The distal pointed portions 456a may have a reduced surface area compared to the surface area of a distal end 451a of an impactor 450a lacking the impact force focusing features. In some examples, the distal pointed portions 456a may include respective flattened regions or may be relatively blunt to prevent damage to healthy tissue in the body lumen. In some examples, such impact force focusing features may be particularly advantageous for loosening, cutting, or tearing treatment-resistant occlusions, such as occlusions formed from fibrotic tissue.

FIG. 4B depicts an alternative example of impact force focusing features that can be included in an impactor 450b of a catheter. In some examples, each impact force focusing features may include spaced-apart flat regions 455b arrayed around the circumference of the distal end 451b. The flat regions 455b may be spaced apart from one another by material-removed region 456b. Like the impact force focusing features shown in FIG. 4A, removing material in the material-removed regions 456b may result in the flat regions 455b having a reduced surface area of the compared to a distal end 451b of an impactor 450b lacking impact force focusing features, such that the flat regions provide increased force to occlusions compared to the distal end of an impactor lacking impact force focusing features. In some examples, the impact force focusing features of FIG. 4B may be particularly advantageous for pulverizing and clearing occlusions, such as calcific occlusions. As such, the impact force focusing features of FIG. 4B may be more suitable for calcific (harder) occlusions than the impact force focusing features of FIG. 4A, which may be more suitable for fibrotic (softer) occlusions.

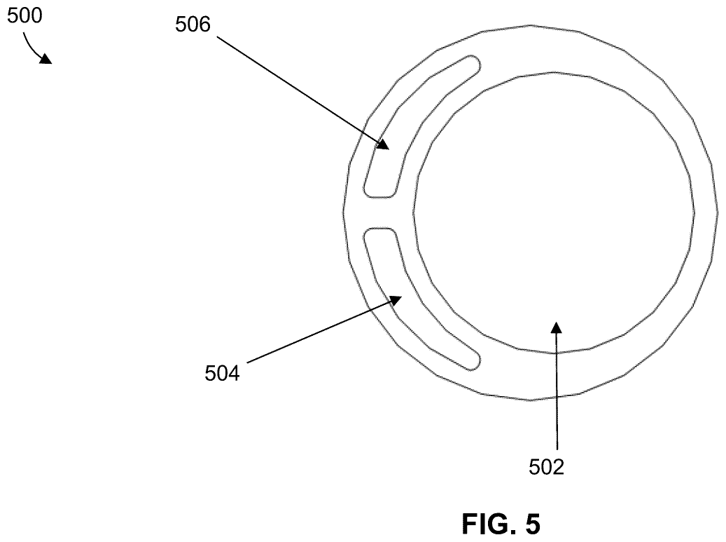

As mentioned above, the elongate body of the catheter may feature various lumens for carrying fluid and other elements between a proximal portion of the catheter (e.g., the handle 200 shown in FIG. 2) and a distal portion of the catheter. FIG. 5 illustrates a cross-sectional view of the elongate body 500 of an exemplary catheter, such as the catheter 10 of FIG. 1, showing the various lumens extending longitudinally through the material of the elongate body. In some examples, one or more of the lumens extend through the entire length of the elongate body 500 (i.e., the entire length of the elongate body between ports included in the proximal handle of the catheter and outlets disposed proximate the distal end of the elongate body). Additionally, or alternatively, one or more of the lumens of the catheter may extend through only a portion of the length of the elongate body 500.

In some examples, the elongate body 500 includes a guide wire lumen 502 sized to receive a guide wire. The guide wire lumen 502 may include an inlet located in a proximal handle of the catheter (e.g., the guide wire port 222 shown in FIG. 2) and an outlet in fluid communication with the body lumen. In some examples, the guide wire lumen 502 additionally functions as an aspiration lumen for evacuating spent fluid and debris from the body lumen. However, in other examples the elongate body 500 includes a separate aspiration lumen and a separate inlet and outlet associated with the aspiration lumen. The aspiration lumen may include an inlet included in the proximal handle of the catheter (e.g., an aspiration port) and an outlet in fluid communication with the body lumen. In some examples, the elongate body additionally includes a flush lumen 504 for flowing fluid into the body lumen through a flush port in the proximal handle of the catheter. The flush lumen 504 may include an outlet in fluid communication with the body lumen and an inlet (e.g., the flush port 224 shown in FIG. 2) included in the distal handle of the catheter. In some examples, the elongate body includes an inflation lumen 506 for flowing fluid into the enclosure and evacuating fluid out of the enclosure. The inflation lumen 506 may include an inlet included in the proximal handle of the catheter (e.g., the inflation port 226 shown in FIG. 2) and an outlet in fluid communication with the volume of the enclosure. In some examples, the outlet of the inflation lumen 506 is an aperture through the material of the elongate body 500 in a region of the body surrounded by the enclosure that permits fluid communication between the inflation lumen 506 and the volume inside the enclosure.

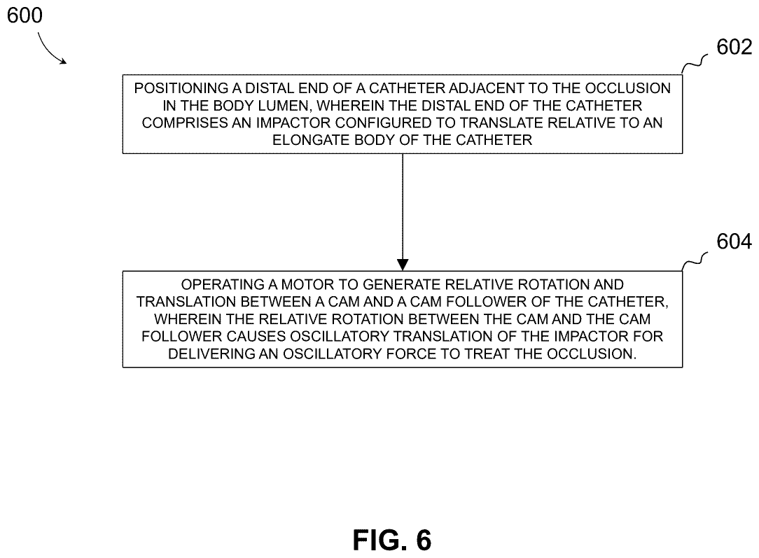

FIG. 6 illustrates a flowchart of an exemplary method of treating an occlusion in a body lumen using a lesion crossing catheter system, such as the catheter system 100 shown in FIG. 1 with any of the features described herein. As described above, such catheters may be particularly effective for treating highly occluded vessels in vasculature, such as CTOs. The catheter system may include a catheter having a handle, an elongate body, and an impactor. The catheter may further include an actuator configured to axially translate the impactor in a distal-proximal direction relative to the elongate body to deliver direct mechanical forces to an occlusion. The catheter may further include an enclosure surrounding at least a portion of the actuator and at least a portion of the impactor. In some examples, the catheter includes a power source (e.g., such as one or more batteries), a motor (e.g., a DC geared motor), and control circuit, which may be housed in a proximal handle of the catheter. In some examples, the handle of the catheter includes various ports for carrying fluid (and, optionally, a guide wire) from the proximal handle to a distal portion of the catheter. For instance, the handle may include a guide wire port, an aspiration port, an inflation port, and a fluid port, and the elongate body may include corresponding lumens for carrying fluids and the guide wire received through the respective ports.

In some examples, step 602 of method 600 includes positioning a distal end of a catheter adjacent to the occlusion in the body lumen. In some examples, positioning the distal end adjacent to the occlusion in the body lumen includes introducing a distal portion of the catheter into the body lumen (e.g., at least a distal portion of the elongate body, the impactor, and the actuator), and advancing the distal portion until the impactor is adjacent to the occlusion in the body lumen. The distal end of the impactor may be in contact with or spaced from the occlusion. The catheter may be advanced until the distal end of the impactor contacts the occlusion (which the user may perceive as increased resistance) and then moved proximally so that the distal end of the impactor is spaced from the occlusion but still close enough to the occlusion that the impactor contacts the occlusion when the oscillator translates distally. In some examples, the enclosure of the catheter is in a collapsed or folded state to reduce a crossing diameter of the catheter when the catheter is positioned within the body lumen. In some examples, insertion of the catheter into the body lumen is facilitated by a guide wire. For instance, introducing the catheter into the body lumen may include advancing the guide wire from an entry site on a patient (e.g., an artery in the groin area of the leg) to the target region of a vessel (e.g., a region having fibrotic occlusions or CTOs that need to be broken up), and advancing the catheter into the body lumen over the guide wire until the distal end of the catheter is positioned adjacent to the occlusion. Optionally, a user of the catheter may track the position of the catheter within the body lumen by use of real-time and/or static imaging devices, including x-ray imaging, intravascular ultrasound (IVUS), optical coherence tomography (OCT), radiofrequency (RF) navigation, and other such techniques.

In some examples, when the distal end of the catheter is positioned adjacent to (contacting or spaced from) the occlusion, the enclosure is filled with fluid. Accordingly, in some examples, the method 600 further includes inflating an enclosure of a catheter with a fluid (e.g., an isotonic fluid such as saline and/or saline mixed with an image contrast agent). In some examples, filling the enclosure with conductive fluid causes the enclosure to inflate such that the outer walls of the enclosure contact the walls of the body lumen and/or lesions inside the body lumen. In some examples, inflating the enclosure positions the impactor of the catheter in approximately the center of the body lumen and secures the distal end of the catheter in the body lumen.

After inflating the enclosure of the catheter, a user may begin treating the occlusion with oscillatory translation of the impactor. Oscillatory translation of the impactor may be driven by way of an actuator (e.g., the actuator 301 shown in FIGS. 3A-3F above) configured to axially translate the impactor relative to the elongate body of the catheter. The actuator may translate mechanical force applied by a motor into the oscillatory translation of the impactor. In some examples, and as described above, the actuator includes an assembly of various components, such as a cam body, including a cam, a rotatable hub including cam follower, a biasing member, thrust bearings, an anti-rotational feature, and other components described above. In some examples, relative rotation between the cam body and the rotatable hub causes the axial translation of the impactor's distal end. Repeated relative rotation between the cam body and rotatable hub may cause the impactor to translate in an oscillatory manner to deliver a series of mechanical forces directly to an occlusion. For instance, cam body may include a cam, and the rotatable hub may include a cam follower extending from the hub and configured to move along the cam. The cam may include a ramp portion and a step portion, and moving the projection past the step portion causes axial translation of the cam and the impactor relative to the cam follower and the elongate body. In such examples, operating the motor to generate relative rotation between the cam and the cam follower may cause the cam follower to move along the surface of the cam, and oscillatory translation of the impactor occurs as the cam follower repeatedly climbs a ramp portion of the cam and moves past a step portion of the cam.

In some examples, step 604 of method 600 includes operating a motor to generate relative rotation and translation between a cam and a cam follower of the catheter. Operating the motor may include inputting a user input on an interface of the catheter, such as a power switch included in the handle of the catheter. In some examples, the motor is configured to rotate a shaft of the catheter, the shaft extending within the elongate body of the catheter. In some examples, the motor is operably coupled to the shaft by way of a gear train, including one or more gears configured to rotate the shaft responsive to operation of the motor. In some examples, the shaft is coupled to one or more components of the actuator, such as either a cam body or a rotatable hub, such that rotation of the shaft causes relative rotation of the cam and cam follower to cause the oscillatory translation of the impactor. In a particular example, the shaft is coupled to the rotatable hub, such that operation of the motor generates relative rotation between the rotatable hub and the cam body to translate the impactor to deliver mechanical forces to treat an occlusion.

In some examples, the motor and/or power source is in communication with a control circuit configured to control aspects of the operation of the catheter. In some examples, the control circuit may be configured to initiate oscillatory translation of the impactor responsive to a user input on the catheter (e.g., by supplying power from the power source to the motor). Additionally, or alternatively, the control circuit may be configured to automatically terminate oscillatory translation of the impactor. For instance, the control circuit may be configured to cease supplying power from the power source to the motor to terminate operation of the motor after a certain period of time (e.g., after an amount of time that approximates the duration of a typical treatment with the catheter), or when the control circuit determines that the power source is low on power.

In some examples, the impactor is configured to oscillate at a frequency between 5 Hz and 400 Hz. However, greater or lesser oscillation frequencies may also be used during a treatment with the catheter, and the number, magnitude, or frequency of the oscillatory translation of the impactor may be controlled by a user of the catheter during a treatment. For instance, the user may control the oscillation frequency via inputs on a user interface of the catheter (e.g., one or more power switches included in a handle of the catheter). In some examples, the control circuit of the catheter may be configured to maintain a stable oscillation frequency during the duration of a treatment. However, in some examples, the control circuit may be configured to change the oscillation frequency during a treatment (e.g., according to a predetermined schedule of oscillation frequencies, or in response to an input from a user of the catheter).

The method 600 may additionally include maneuvering the impactor of the catheter inside the body lumen to drive the impactor into the occlusion. For instance, a user of the catheter may maneuver a distal end of the impactor into an occlusion (e.g., a distal end including one or more impact force focusing features) to contact the occlusion and apply mechanical forces to the occlusion. Application of the initial mechanical force may cause an initial puncture of the occlusion (e.g., to penetrate a fibrous cap of an occlusion). Further application of force may cause the impactor to drill into the occlusion, creating a small channel in the occlusion. The user may then proceed to maneuver the oscillating impactor through the occlusion to break up and clear the occlusion.

After a first series of mechanical forces are delivered to the occlusion, the catheter can be repositioned or advanced further in the body lumen to continue treatment. The progress of the procedure may be monitored by x-ray and/or fluoroscopy. As the occlusion is broken up or penetrated by mechanical forces from the oscillating impactor, the impactor can be advanced farther into the lesion, and treatment can be repeated until the total occlusion is cleared or until the diameter of the vessel permits the placement of a second treatment device having a larger profile. For example, the enlarged channel can receive a different catheter, such as an IVL. Catheters of this type are described in U.S. Pat. No. 8,747,416 and U.S. Publication No. 2019/0150960, cited above. Once the lesion has been sufficiently treated, the catheter can be withdrawn from the body lumen.

In some examples, the treatment may be conducted in one or more stages or phases. For instance, a physician may initially position the catheter near a first portion of an occlusion and apply mechanical forces to the first portion of the occlusion using the oscillating impactor. If the occlusion is not cleared by a first round of treatment, the user may reposition the catheter further along the length of the body lumen and treat a second portion of the occlusion using mechanical forces from the vibrating guide wire and/or elongate body. Once the occlusion has been sufficiently treated, the enclosure may optionally be inflated further or deflated, and the catheter and guide wire may be withdrawn from the patient.

In some examples, a user of the catheter may terminate the oscillatory translation via a user input (or the termination of a user input) on a handle of the catheter. For instance, the user may press a button on the handle to terminate the oscillatory translation of the impactor or may cease pressing a button on the handle of the catheter to terminate the oscillatory translation of the impactor. Once the impactor ceases oscillatory translation, the distal portion of the catheter may be removed from the body lumen. Removing the catheter from the body lumen may include deflating the enclosure of the catheter. In some examples, the enclosure may be deflated by removing fluid from the balloon via an inflation lumen that evacuates the fluid via an inflation port in the handle. In some examples, at least a portion of the catheter is a disposable or single-use catheter. For instance, the entire catheter may be single-user or disposable, or the distal portion of the catheter may be single-use or disposable. In such examples, the catheter may be discarded after a single treatment or after a certain number of treatments.

Although the catheter devices described herein have been discussed primarily in the context of treating occlusions in vasculature, such as chronic and resistant occlusions and CTOs, the catheters herein can be used for a variety of occlusions, such as occlusions in the peripheral vasculature (e.g., above-the-knee, below-the-knee, iliac, carotid, etc.). For further examples, similar designs may be used for treating soft tissues, such as cancer and tumors (i.e., non-thermal ablation methods), blood clots, fibroids, cysts, organs, scar and fibrotic tissue removal, or other tissue destruction and removal. Catheter designs could also be used for neurostimulation treatments, targeted drug delivery, treatments of tumors in body lumens (e.g., tumors in blood vessels, the esophagus, intestines, stomach, or vagina), wound treatment, or non-surgical removal and destruction of tissue.

In one or more examples, the catheters described herein could also be used for tissue engineering methods, for instance, for mechanical tissue decellularization to create a bioactive scaffold in which new cells (e.g., exogenous or endogenous cells) can replace the old cells; introducing porosity to a site to improve cellular retention, cellular infiltration/migration, and diffusion of nutrients and signaling molecules to promote angiogenesis, cellular proliferation, and tissue regeneration similar to cell replacement therapy. Such tissue engineering methods may be useful for treating ischemic heart disease, fibrotic liver, fibrotic bowel, and traumatic spinal cord injury (SCI). For instance, for the treatment of spinal cord injury, the devices and assemblies described herein could facilitate the removal of scarred spinal cord tissue, which acts like a barrier for neuronal reconnection, before the injection of an anti-inflammatory hydrogel loaded with lentivirus to genetically engineer the spinal cord neurons to regenerate.

The elements and features of the exemplary catheters discussed above may be rearranged, recombined, and modified, without departing from the present invention. Furthermore, numerical designators such as “first,” “second,” “third,” “fourth,” etc., are merely descriptive and do not indicate a relative order, location, or identity of elements or features described by the designators. Accordingly, numerical designators of various elements and features are not intended to limit the disclosure and may be modified and interchanged without departing from the subject invention.

It should be noted that the elements and features of the example catheters illustrated throughout this specification and drawings may be rearranged, recombined, and modified without departing from the present invention. For instance, while this specification and drawings describe and illustrate catheters having several enclosure and emitter designs, the present disclosure is intended to include catheters having a variety of enclosure and emitter configurations. The number, placement, and spacing of the shock wave emitters can be modified without departing from the subject invention. Further, the number, placement, and spacing of enclosures of catheters can be modified without departing from the subject invention.

It should be understood that the foregoing is only illustrative of the principles of the invention, and that various modifications, alterations, and combinations can be made by those skilled in the art without departing from the scope and spirit of the invention. Any of the variations of the various catheters disclosed herein can include features described by any other catheters or combination of catheters herein. Furthermore, any of the methods can be used with any of the catheters disclosed. Accordingly, it is not intended that the invention be limited, except as by the appended claims.

1. A catheter for treating an occlusion in a body lumen, the catheter comprising:
an elongate body;
a rotatable shaft disposed within the elongate body;
an impactor forming a distal end of the catheter and configured to translate relative to the elongate body for impacting an occlusion in the body lumen; and
an actuator configured to translate the impactor, the actuator comprising:
a cam and cam follower operatively coupled to the rotatable shaft such that rotation of the rotatable shaft causes relative rotation and translation between the cam and cam follower, wherein the impactor is coupled to the cam or the cam follower such that the impactor translates in conjunction with the cam or the cam follower to impact the occlusion in the body lumen.

2. The catheter of claim 1, further comprising an enclosure surrounding at least a portion of the elongate body.

3. The catheter of claim 1, wherein the impactor is coupled to the cam and translates in conjunction with the cam.

4. The catheter of claim 3, wherein the cam is coupled to the impactor via a coupler.

5. The catheter of claim 1, further comprising a motor operable to rotate the shaft.

6. The catheter of claim 5, wherein the shaft is coupled to the motor via a gear train.

7. The catheter of claim 1, wherein the cam follower is coupled to the shaft and rotates in conjunction with the shaft.

8. The catheter of claim 1, wherein the cam follower projects from a rotatable hub.

9. The catheter of claim 1, wherein:
the cam comprises at least one ramp portion and at least one step portion, and
the impactor translates in a distal direction when the cam follower moves past the at least one step portion from the at least one ramp.

10. The catheter of claim 9, wherein the cam comprises multiple ramp portions and multiple step portions.

11. The catheter of claim 9, wherein movement of the cam follower along the ramp portion causes an increase in biasing force of a biasing member.

12. The catheter of claim 11, wherein the biasing member is a coil spring.

13. The catheter of claim 1, wherein the actuator is configured to translate the impactor up to 0.75 millimeters.

14. The catheter of claim 1, wherein the actuator is configured to translate the impactor at least 0.25 millimeters.

15. The catheter of claim 1, wherein a frequency of oscillatory translation of the impactor is up to 400 Hz.

16. The catheter of claim 1, wherein a frequency of oscillatory translation of the impactor is at least 50 Hz.

17. The catheter of claim 1, wherein the distal tip of the impactor comprises one or more impact force focusing features.

18. The catheter of claim 1, further comprising a handle, wherein a motor is housed within the handle for rotating the shaft.

19. The catheter of claim 18, wherein the motor is battery powered.

20. A method of treating an occlusion in a body lumen, the method comprising:
positioning a distal end of a catheter adjacent to the occlusion in the body lumen, wherein the distal end of the catheter comprises an impactor configured to translate relative to an elongate body for impacting an occlusion in the body lumen; and
operating a motor to generate relative rotation and translation between a cam and a cam follower of the catheter, wherein the relative rotation and translation between the cam and the cam follower causes the impactor to translate in conjunction with the cam or the cam follower to impact the occlusion in the body lumen.

21. The method of claim 20, further comprising inflating an enclosure of the catheter.

22. The method of claim 21, wherein the enclosure is inflated prior to operating the motor to stabilize the distal end of the catheter within the body lumen.

23. The method of claim 20, wherein the impactor is coupled to the cam and translates in conjunction with the cam.

24. The method of claim 20, wherein the catheter comprises a shaft extending within the elongate body that is rotated by the motor.

25. The method of claim 20, wherein:
the cam comprises a contact surface,
the cam follower comprises a rotatable hub and a projection extending from the hub and configured to move along the contact surface, and
operating the motor to generate relative rotation between a cam and a cam follower causes the projection to move along the contact surface.

26. The method of claim 25, wherein:
the contact surface comprises at least one ramp portion and at least one step portion, and
the impactor is configured to translate in a distal direction when the projection moves past the at least one step portion.

27. The method of claim 20, wherein:
the elongate body comprises a guide wire lumen configured to receive a guide wire, and
positioning the distal end of the catheter adjacent to the occlusion in the body lumen comprises:
introducing a guide wire into the body lumen, and
advancing the distal end of the catheter into the body lumen over the guide wire.

28. The method of claim 20, wherein positioning the distal end of the catheter adjacent to the occlusion comprises advancing the distal end of the catheter distally in the body lumen until the distal end contacts the occlusion.

29. The method of claim 20, wherein the occlusion comprises fibrotic tissue and a distal end of the impactor comprises at least one force focusing feature that comprises a sloping portion that leads to a pointed portion.

30. The method of claim 20, wherein the occlusion comprises calcific tissue and a distal end of the impactor comprises a plurality of spaced-apart regions.

31. A catheter for treating an occlusion in a body lumen, the catheter comprising:
an elongate body;
a shaft disposed within the elongate body;
an impactor forming a distal end of the catheter and configured to translate relative to the elongate body for impacting an occlusion in the body lumen; and
an actuator configured to translate the impactor;
a handle located at a proximal end of the catheter;
a motor located in the handle and connected to the actuator; and
a battery located in the handle for powering the motor.
